(19) United States
(12) Patent Application Publication
(10) Pub. No.: US 2016/0051567 A1
(43) Pub. Date: Feb. 25, 2016

(54) PREVENTION AND TREATMENT OF KIDNEY DAMAGE

(71) Applicant: METSELEX, INC., Minneapolis, MN (US)

(72) Inventors: Clifford STEER, Eagan, MN (US); Sandeep GUPTA, Minneapolis, MN (US)

(73) Assignee: Metselex, Inc., Minneapolis, MN (US)

(21) Appl. No.: 14/780,288

(22) PCT Filed: Mar. 27, 2014
(86) PCT No.: PCT/US2014/032040

§ 371 Date: Sep. 25, 2015

Related U.S. Application Data
(60) Provisional application No. 61/805,649, filed on Mar. 27, 2013.

(51) Int. Cl. A61K 31/575; A61K 9/00

(52) U.S. Cl. CPC A61K 31/575; A61K 9/0019

(57) ABSTRACT

A method of treating or preventing kidney injury includes administering to a patient an effective amount of bile acid, a salt thereof, an analog thereof, or a combination thereof. Methods of preventing or retarding, reversing or abolishing the onset of kidney injuries are discussed. This is achieved through the administration of a bile acid, a salt of the bile acid, an analog of the bile acid or any combinations of these compounds. The bile acid abolishes or interferes or down-regulates metabolic pathways leading to the onset of kidney injury. The bile acid also activates metabolic pathways leading to the slowing or reversing or complete abolishment of the progression of acute kidney injury.

### BACKGROUND OF THE INVENTION

Kidneys are acutely injured when deprived of nutrients and exposed to nephrotoxins. Acute kidney injury (AKI) is a disease that has reached epidemic proportions and has grave short- and long-term consequences on patient health and cost of care. Even kidneys that regain normal function following AKI have persistent maladaptive alterations that may result in a higher incidence of hypertension and chronic kidney disease. Even in situations where the onset of AKI is predictable, such as to perioperative kidney injury, none of the current therapies can prevent AKI. Thus, there is a critical need to develop therapies for the prevention of AKI.

### SUMMARY OF THE INVENTION

A method of treating or preventing kidney injury includes administering to a patient an effective amount of bile acid, a salt thereof, an analog thereof, or a combination thereof. The present invention provides a method of preventing or treating kidney damage through the administration of any forms of bile acids. The administering step involves administering, through various means, an amount of tauroursodeoxycholic acid (TUDCA) or other bile acids that is effective in providing the necessary pharmacological benefit. The mode of administering TUDCA includes, but is not limited to, intravenously, parenterally, orally or intramuscularly or any combination of these methods thereof.

### BRIEF DESCRIPTION OF THE FIGURES

FIG. 1 is a graph showing blood urea levels in mice treated with TUDCA and untreated mice.

FIG. 2 shows histological injury scores for mice treated with TUDCA and untreated mice.

FIG. 3 shows the percentage of TUNEL-positive cells in mice treated with TUDCA and untreated mice.

FIG. 4 shows caspase-9 activation results following AKI for mice treated with TUDCA and untreated mice.

FIG. 5 shows extracellular signal-related kinase (ERK) and c-Jun N-terminal kinase (JNK) results for mice treated with TUDCA and untreated mice.

FIG. 6 shows cytotoxicity and viability results for mice treated with varying levels of TUDCA and untreated mice.

FIG. 7 shows caspase activation following cryoinjury and treatment with TUDCA in primary human renal proximal tubule epithelial (RPTE) cells.

FIG. 8 shows phosphorylated ERK1/2 protein results for primary human RPTE cells treated with TUDCA following cryoinjury.

### DETAILED DESCRIPTION OF THE INVENTION

The current disclosure details a method for treating kidney injuries in their various forms. In the existing state of medical arts there are few effective therapies that would to ameliorate, reverse or prevent kidney injury. Herein, a “patient” includes a human or any mammal.

The disclosure also details a study to determine the protective properties of TUDCA against AKI. Two models of AKI to simulate commonly encountered clinical scenarios were selected: 1) the warm ischemia reperfusion model of AKI in rats recapitulates clinical AKI in the native kidney due to poor perfusion; and 2) the cellular model of cryopreservation injury recapitulates cryopreservation-associated AKI in the donor kidney. A cellular model was chosen in preference to in vivo models of cryopreservation injury to prevent systemic and donor factors such as immunity, inflammation, and donor age from confounding the results.

Following AKI, cells die either immediately by necrosis or over hours to days by apoptosis, or programmed cell death. Cells under stress resist death by upregulating survival pathways. AKI can be prevented under experimental conditions by upregulating survival pathways by pro-survival molecules such as Survivin or by ischemic preconditioning. Similarly, anti-apoptotic molecules have been shown to prevent AKI in animal models. However, these experimental approaches are limited in their translational potential by toxicity. Therefore, an ideal therapy for prevention of AKI should be nontoxic, pro-survival, and anti-apoptotic.

The liver may provide clues for developing such a therapy for AKI. Liver cells are exposed to toxic compounds and have well-developed cytoprotective mechanisms. Protection by ursodeoxycholic acid (UDCA) and its taurine conjugate, tauroursodeoxycholic acid (TUDCA), has been studied. UDCA and TUDCA prevent cell death by stabilizing cell membranes, inhibiting apoptosis, and upregulating survival pathways. Furthermore, protection by UDCA and TUDCA extends beyond liver to other cells in the body. For example, hibernating animals such as black bears have high blood levels of UDCA, which prevents cell death under low nutrient conditions encountered during long periods of hibernation. In contrast, humans have very low blood levels of UDCA. Black bear bile has been used in traditional Chinese medicine for more than 3000 years; and western medicine is increasingly recognizing the therapeutic value of UDCA and TUDCA. UDCA and TUDCA have been used effectively for treating human liver diseases and in experimental models of acute injury such as myocardial infarction, stroke, and spinal cord injury. Furthermore, several studies have shown UDCA and TUDCA to be safe for animal and human applications, making them attractive molecules from a translational standpoint.

AKI is often predictable in clinical situations such as following surgery; exposure to nephrotoxic medications; and donor nephrectomy during cryopreservation. However, no current state of the art therapy can prevent AKI. Our vision in planning the described studies was to develop a therapy with high translational potential that can be administered for prevention of AKI. Thus, studies summarized herein tested the hypothesis that TUDCA can prevent AKI. We chose TUDCA over UDCA because of its higher solubility at physiological pH, a characteristic that permits rapid parenteral administration in high doses and avoids precipitation during cryopreservation of donor kidneys. Accordingly, the studies described determined the efficacy and mechanisms of action of TUDCA in a rat model of AKI and a human kidney cell culture model of cryopreservation injury.

Warm ischemia reperfusion injury for 45 minutes at 37° C. produced a very consistent injury in the rat model. The peak rise in blood urea occurred 24 hours following bilateral renal artery clamping in both the vehicle (control)—and TUDCA-treated rats. As compared to rats treated with vehicle, rats treated with TUDCA had significantly less peak elevation in blood urea following AKI. Blood urea values subsequently declined until day 4, when they plateaued in the vehicle group, while they continued to improve in the TUDCA group until the day of euthanasia. Thus, TUDCA reduced the severity of AKI without postponing its onset.

The functional protection against AKI by TUDCA was supported by less severe histological injury seen in kidneys of TUDCA-treated rats. The deep cortex, where the S3 segment of the proximal tubule is located and which sustains maximum injury following ischemia-reperfusion, was better preserved in the TUDCA group. Furthermore, TUDCA provided protection against apoptosis following AKI. There were significantly less TUNEL-positive cells (cells indicative of DNA fragmentation) in the superficial and deep cortices and in the outer strip of the outer medulla in the TUDCA-treated rats as compared to the vehicle-treated rats. Based on the morphological appearance, the TUNEL-positive cells were present exclusively in the proximal tubules. We did not find apoptotic cells in the distal tubules following AKI as reported by other investigators.

Activation of caspase-9, which represents the mitochondrial pathway of apoptosis, was significantly inhibited by TUDCA following AKI. TUDCA has been shown to inhibit the mitochondrial pathway of apoptosis in primary hepatocytes, neurons, and in animal models of ischemic injury such as stroke. Similar to the mitochondrial pathway of apoptosis, the endoplasmic reticulum-stress (ER-stress) pathway of apoptosis plays an important role in the pathogenesis of glomerular, tubular, and interstitial kidney diseases. In particular, following ischemic kidney injury, investigators have shown activation of the ER-stress pathway of apoptosis and protection by its inhibitors. In contrast to the mitochondrial pathway, in our model, TUDCA did not have any effect on the ER-stress and death receptor pathways of apoptosis. This is surprising in light of recent studies demonstrating the ability of TUDCA to reduce ER-stress induced caspase-12 activation.

Mitogen-activated protein kinases (MAPKs) constitute important survival pathways in mammals, which include c-Jun N-terminal kinase (JNK), p38, and extracellular signal-related kinase (ERK). Activation of JNK and p38 has been shown to facilitate cell death, while activation of ERK1/2 promotes cell survival following acute injury. Furthermore, ischemic preconditioning of kidneys, which is protective against AKI, acts through activation of ERK1/2. In our model of AKI, TUDCA increased ERK1/2 levels in two out of three rats. TUDCA had no effect on JNK and p38 pathways. Although this finding is encouraging, it needs further confirmation before one can conclude that activation of ERK1/2 by TUDCA following AKI plays a protective role.

Similar to native kidneys, AKI in the donor kidney is a significant clinical problem. Cessation of blood supply following harvesting results in AKI. To minimize this risk, donor kidneys are currently cryopreserved in specialized solutions such as University of Wisconsin cryopreservative solution (contains 100 mM potassium lactobionate, 25 mM KH₂PO₄, 5 mM MgSO₄, 30 mM raffinose, 5 mM adenosine, 3 mM glutathione, 1 mM allopurinol and 50 g/L hydroxyethyl starch). Although a major advancement in the field, the current state of the art cryopreservation techniques still result in significant graft injury. Cryopreservation injury to the donor kidney leads to increased incidence of delayed graft function, acute and chronic rejection, and poor short- and long-term graft outcome. Furthermore, the current cryopreservation time in the United States has remained long at approximately 21 hours. Therefore, there is a pressing need to improve the current cryopreservation techniques. The current studies were performed to set the stage for developing improved cryopreservation solutions for clinical use.

Kidney cells die by necrosis during cryopreservation and by apoptosis during warm reperfusion following transplantation. There is activation of apoptosis pathways during cryopreservation, and survival pathways play an important role in resisting cell death. The current study investigated the efficacy and mechanisms of action of TUDCA to against cryopreservation injury. TUDCA was not cytotoxic to RPTE cells in concentrations up to 600 μM (micromolar) and it did not decrease cell viability in any of the tested concentrations. TUDCA has been shown to be safe in concentrations ranging from 100 nM up to 5 mM in cell culture experiments. Chosen concentrations ranged from 100 μM to 150 μM of TUDCA.

Caspase-3 is activated following cryopreservation of cells and a caspase inhibitor provides protection. Similar to the published literature, caspase-3 was consistently activated following cryopreservation injury to the RPTE cells, and was found to be inhibited by TUDCA in a dose-dependent fashion. Similarly, there was activation of the mitochondrial pathway of apoptosis in our model of cryopreservation injury, and 100 μM and 150 μM of TUDCA provided protection. This is an advancement of the previously known anti-apoptotic properties of TUDCA in models of warm ischemia-reperfusion injury. Unlike the mitochondrial pathway of apoptosis, there was no activation of the ER-stress and death receptor pathway of apoptosis following cryopreservation injury in cases where TUDCA was administered.

There was activation of ERK1/2 survival pathways by both 100 μM and 150 μM of TUDCA following cryopreservation injury to the RPTE cells. This is similar to upregulation of ERK1/2 seen in two of three rats in the in vivo model of AKI. This is the first report of activation of ERK1/2 pathways by TUDCA following cryopreservation injury. Similar to the in vivo model, there was no effect of TUDCA on the JNK and p38 pathways following cryopreservation injury.

In conclusion, TUDCA was shown to be protective in the rat model of ischemia-reperfusion induced AKI and cellular model of cryopreservation injury. It provided protection in the tested models of AKI by inhibiting the mitochondrial pathway of apoptosis and upregulating ERK1/2 survival pathways. Results of this study and a proven safety profile of TUDCA in humans will open the door for conducting human feasibility studies in patients with AKI, an important area of investigation that currently lacks effective therapy. We anticipate administration of TUDCA prior to precipitating events will prevent AKI in humans through either the down-regulation of any metabolic pathways that lead to kidney injury, or by the up-regulation of metabolic pathways that slow or reverse the progression of kidney injury.

The methods of the current invention are associated with the utilization of a hydrophilic bile acid, its salts thereof and analogs thereof, and combinations thereof. These bile acids are more hydrophilic than TUDCA's isomer chenodeoxycholic acid to (CDCA). The hydrophilic bile acids also include ursodeoxycholic acid (UDCA).

Analogs of TUDCA include, among others, conjugated derivatives of bile acids such as nor-ursodeoxycholic acid, glycol-ursodeoxycholic acid, ursodeoxycholic acid 3-sulfate, ursodeoxycholic acid 7-sulfate, and ursodeoxycholic acid 3,7-sulfate.

These hydrophilic bile acids are used in amounts effective to treat kidney injury by either or both prophylactic or therapeutic treatments. Treatment involves prevention of onset or retardation or complete reversal of any or all symptoms or pharmacological or physiological or neurological or biochemical indications associated with kidney injury. Treatment can begin either with the earliest detectable symptoms or established symptoms of kidney injury.

The “effective” amount of the compound thereof is the dosage that will prevent or retard or completely abolish any or all pathophysiological features associated with various stages (late or end) kidney injury (sporadic or familial).

The hydrophilic bile acids can be combined with a formulation that includes a suitable carrier. Preferably, the compounds utilized in the formulation are of pharmaceutical grade. This formulation can be administered to the patent, which includes any mammal, in various ways which are, but not limited to, oral, intravenous, intramuscular, nasal, or parental (including, and not limited to, subcutaneous, intramuscular, intraperitoneal, intravenous, intrathecal, intraventricular, and direct injection into the brain or spinal tissue).

Formulations can be prepared and presented to the patient by any of the methods in the realm of the art of pharmacy. These formulations are prepared by mixing the biologically-active hydrophilic bile acid into association with compounds that include a carrier. The carrier can be liquid, granulate, solid (coarse or finely broken), liposomes (including liposomes prepared in combination with any non-lipid small or large molecule), or any combination thereof.

The formulation in the current invention can be furnished in distinct units including, but not limited to, tablets, capsules, caplets, lozenges, wafers, and troches with each unit containing specific amounts of the active molecule for treating acute kidney injury of any form. The active molecule can be incorporated in a powder, encapsulated in liposomes, in granular form, in a solution, in a suspension, in a syrup, in any emulsified form, in a drought or in an elixir.

Tablets, capsules, caplets, pills, troches, etc. that contain the biologically-active to hydrophilic bile acid can contain binder (including, but not limited to, corn starch, gelatin, acacia, and gum tragacanth), an excipient agent (including but not limited to dicalcium phosphate), a disintegrating agent (including but not limited to corn starch, potato starch, and alginic acid) a lubricant (including but not limited to magnesium stearate), a sweetening agent (including but not limited to sucrose, fructose, lactose, and aspartame), and a natural or artificial flavoring agent. A capsule can additionally contain a liquid carrier. Formulations can be of quick-, sustained-, or extended-release type.

Syrups or elixirs can contain one or several sweetening agents, preservatives, crystallization-retarding agents, solubility-enhancing agents, etc.

Any or all formulations containing the biologically-active hydrophilic bile acids can be included into the food (liquid or solid or any combination thereof) of the patient. This inclusion can either be an additive or supplement or similar or a combination thereof.

Parenteral formulations are sterile preparations of the desired biologically-active hydrophilic bile acid can be aqueous solutions, dispersions of sterile powders, etc., that are isotonic with the blood physiology of the patient. Examples of isotonic agents include, but are not limited to, sugars, buffers (example saline), and any salts.

Formulations for nasal spray are sterile aqueous solutions containing the biologically-active hydrophilic bile acid along with preservatives and isotonic agents. The sterile formulations are compatible with the nasal mucous membranes.

The formulation can also include a dermal patch containing the appropriate sterile formulation with the active agent. The formulation would release the active agent into the blood stream either in sustained or extended or accelerated or decelerated manner.

The formulation can also include a combination of compounds, in any of the afore mentioned formulations designed to traverse the blood-brain barrier.

### Examples

In the following examples, the function of TUDCA in its various forms in arresting or delaying or entirely preventing the onset of acute kidney injury is further characterized. Specifically, TUDCA treatment led to the prevention or reduction of acute kidney injury.

### Materials

Normal human RPTE cells were purchased from Lonza Inc. (Walkersville, Md.). The University of Wisconsin solution was obtained from BTL solutions LLC (Columbia, S.C.). TUDCA and Cell Lytic MT Mammalian Tissue Lysis/Extraction Reagent were to purchased from Sigma (St Louis, Mo.). Protease Inhibitor Cocktail and Bicinchoninic Acid Protein Assay kits were purchased from Pierce Biotechnology Inc. (Rockford, Ill.). 12% Tris-HCl sodium dodecyl sulfate-polyacrylamide gel electrophoresis (SDS-PAGE) pre-cast gel was obtained from Bio-Rad Laboratories Inc. (Hercules, Calif.). Amersham Hybond ECL nitrocellulose membrane, Amersham hyperfilm and peroxidase-labeled anti-mouse/rabbit IgG were purchased from GE Healthcare (Waukesha, Wis.). Anti-phospho-ERK1/2 antibody was purchased from New England BioLabs (Boston, Mass.); anti-phospho-p38 and anti-phospho-JNK from Santa Cruz Biotechnology Inc. (Santa Cruz, Calif.); anti-caspase-8 and anti-caspase-12 from BioVision Inc. (Mountainview, Calif.); anti-caspase-9 antibody from Enzo Life Sciences (Plymouth Meeting, Pa.); and anti-mouse β-actin from Calbiochem (Spring Valley, Calif.). SuperSignal West Femto Maximum Sensitivity Substrate Kit was from Thermo Fisher Scientific (Waltham, Mass.). MultiTox-Glo Multiplex Cytotoxicity Assay and Caspase-9 Glo Assay kits were purchased from Promega Corp. (Madison, Wis.). Apo-One® Homogeneous Caspase-3/7 Assay kit and Caspase-9 Assay kit were purchased from Promega (Madison, Wis.). Male Sprague Dawley rats were purchased from Harlan Laboratories (Indianapolis, Ind.). QuantiChrom™ Urea Assay kit was purchased from BioAssay Systems (Hayward, Calif.), APO-DIRECT™ kit was purchased from BD Pharmingen (San Diego, Calif.). Reflex Clips Applier and Reflex 9 mm Clips were obtained from World Precision Instruments, Inc. (Sarasota, Fla.). 4-0 absorbable sutures were purchased from Ethicon, Johnson and Johnson (Somerville, N.J.).

### In Vivo Experiments

Rat model of AKI: All experiments were performed in accordance with the Institutional Animal Care and Use Committee. Six to eight week old Sprague-Dawley rats were anaesthetized by isoflurane gas, a midline abdominal incision was made, and bilateral renal pedicles were clamped for 45 minutes maintaining body temperature at 37° C. After removing the clamps, the abdomen was closed in two layers by using 4-0 absorbable sutures and Reflex 9 mm clips. Blood samples were obtained daily by tail vein puncture. Blood urea levels were measured by the improved Jung method using the QuantiChrom™ Urea Assay Kit as per manufacturer's protocol. Kidneys were harvested five days following surgery.

Administration of TUDCA: 200 mg/mL of TUDCA (Sigma) was dissolved in phosphate buffered saline at pH 7.5. 400 mg/kg of TUDCA or equal volume of vehicle was administered to rats by daily intraperitoneal injection from three days prior to surgery (day 3) to five days (day 5) following surgery. The TUDCA dose was based on previous studies and its solubility at physiological pH.

Histology and TUNEL assay: 4% paraformaldehyde-fixed, paraffin-embedded 5 μm kidney sections were stained with Periodic acid-Schiff (PAS) stain using standard methods. Histological examination was performed by a renal pathologist in a blinded fashion. Histological injury was scored based on the percentage of tubular cell necrosis, dilation, and cell detachment as per the PAS protocol. Reagents for the PAS assay were purchased from Sigma-Aldrich (St. Louis, Mo.) and the accompanying protocol was used. In brief, the following criteria were used: 0, no abnormality; 1+, changes affecting less than 25% of sample; 2+, changes affecting 25%-50%; 3+, changes affecting 50%-75%; and 4+, changes affecting >75% of the sample. Apoptotic cells were detected by TUNEL assay using an APO-DIRECT™ kit as per manufacturer's protocol. The average injury score and percentage of TUNEL-positive cells from the renal cortex (superficial and deep), medulla (outer and inner), and papilla were counted and calculated separately and averaged to obtain the total score. At least 10 fields (under ×200 magnification) were reviewed at each location.

### Cell Culture Experiments

Cell culture: RPTE cells were grown in Renal Epithelial Cell Basal Medium (REBM) with full supplements at 37° C. in 5% CO₂ incubator as per supplier's instructions. RPTE cells were able to proliferate for 6-8 passages under the culture conditions.

Cryopreservation injury: We have utilized a published cell culture model of cryopreservation injury. In brief, RPTE cells were grown to 80% confluence in the complete medium containing TUDCA or vehicle. The complete medium was then replaced with University of Wisconsin solution containing TUDCA or vehicle. The culture plates were subsequently incubated in a temperature-regulated refrigerator at 4° C. for 48 hours. To simulate warm reperfusion phase of kidney transplantation, University of Wisconsin solution was replaced with complete medium containing TUDCA or vehicle, and cells were cultured for an additional 24 hours at 37° C. We used 100 μM or 150 μM of TUDCA for these experiments.

Viability and Cytotoxicity Assays: Cytotoxicity and viability were determined using MultiTox-Glo Multiplex Cytotoxicity Assay kit as per manufacturer's protocols. RPTE cells were seeded in 96-well culture plates at a density of 1.2×10⁴ cells per well. Subsequently, TUDCA was added to the wells to achieve final concentrations of 15, 150, 300, 450, 600, and 1200 μM. Following 24 hours of culture, to determine viability, 50 μL of GF-AFC Reagent was added to each well. The plates were gently shaken and incubated at 37° C. for 30 minutes in the dark. The cell viability was determined by measuring fluorescence at 400 nm_{Ex}/505 nm_{Em}. Subsequently, to determine cytotoxicity, 50 μL of AAF-Glo Reagent was added to each well. The plates were shaken gently and incubated at room temperature for 15 minutes in the dark. Cytotoxicity was determined by measuring luminescence as per manufacturer's protocol.

### Other Methods

Protein extraction: Frozen kidney tissue was ground in liquid nitrogen using a pestle and mortar. One mL of Tissue Protein Extraction Reagent with lx protease and phosphatase inhibitor was added to the ground kidney tissue (per 30 mg of tissue) or RPTE cells (per 1.2×10⁶ cells). The lysate was incubated at 4° C. for 10 minutes with vigorous shaking and subsequently centrifuged at 4° C. for 10 minutes at 13,000 g. The resultant supernatant was immediately frozen in liquid nitrogen and stored at −80° C. until further analysis. The amount of protein present in the solution was quantified by the Bicinchoninic Acid Protein Assay kit as per manufacturer's protocol.

Western blot analysis: 20 μg of protein was mixed with SDS-PAGE sample buffer, boiled for 5 minutes, electrophoresed on a 12% Tris-HCl SDS-PAGE gel for 1 hour at 200 V, and electroblotted onto a nitrocellulose membrane. The membrane was blocked for 1 hour at room temperature in 1×Tris-buffered saline with 0.1% Tween-20 at pH 7.4 containing 5% dry milk powder. To detect p-ERK1/2, p-JNK, p-p38, caspase-8, caspase-9, and caspase-12, the membranes were incubated overnight with respective primary antibodies at 4° C. and appropriate secondary antibodies at room temperature for one hour. The immunoblot was detected using SuperSignal West Femto Maximum Sensitivity Substrate Kit. To verify equal loading of proteins, the membrane was stripped at room temperature with lx Tris-buffered saline at pH 2.5 for 30 minutes and re-probed with the mouse anti-β-actin antibody and corresponding secondary antibody.

Caspase-3 activity assay: The caspase-3 activity in kidney and RPTE extracts was quantified using Apo-One® Homogeneous Caspase-3/7 Assay kit as per the manufacturer's protocol. In brief, 100 pg of protein in 100 μL of lysis buffer was added to 100 μL of the assay buffer containing non-fluorescent caspase-3 substrate, bis-N-CBZL-aspartyl-L-glutamyl-L-valyl-L-aspartic acid amide (Z-DEVD-R110). The mixture was then incubated for 1 hour at 30° C. during which Z-DEVD-R110 was converted into a fluorescent substrate by the active caspase-3 enzyme. Fluorescence was measured by Spectramax M12 fluorescent plate reader (Molecular Devices) using wavelengths of 485 nm_{Ex}/520 nm_{Em}. The fluorescent signal was expressed in relative fluorescent units.

Caspase-9 Assay: The caspase-9 activity was measured in RPTE cells using Caspase-9 Assay kit. RPTE cells were suspended in 50 μL of chilled Cell Lysis Buffer and incubated on ice for 10 minutes. Subsequently, 50 μL of 2× Reaction Buffer and 5 μL of 1 mM LEHD-AFC substrate was added to each sample and the mixtures were incubated at 37° C. for 2 hours. The caspase-9 activity was quantified by measuring luminescence.

### Statistical Analysis

Data were expressed as mean and standard deviation unless otherwise stated. The differences between normally distributed data were analyzed by independent Student's T-test. Nonparametric unpaired Mann-Whitney test was used if the data was not normally distributed. Multiple group comparisons were performed using ANOVA with post-test according to Bonferroni. A p value of less than 0.05 was considered statistically significant.

### Results

### In Vivo Experiments

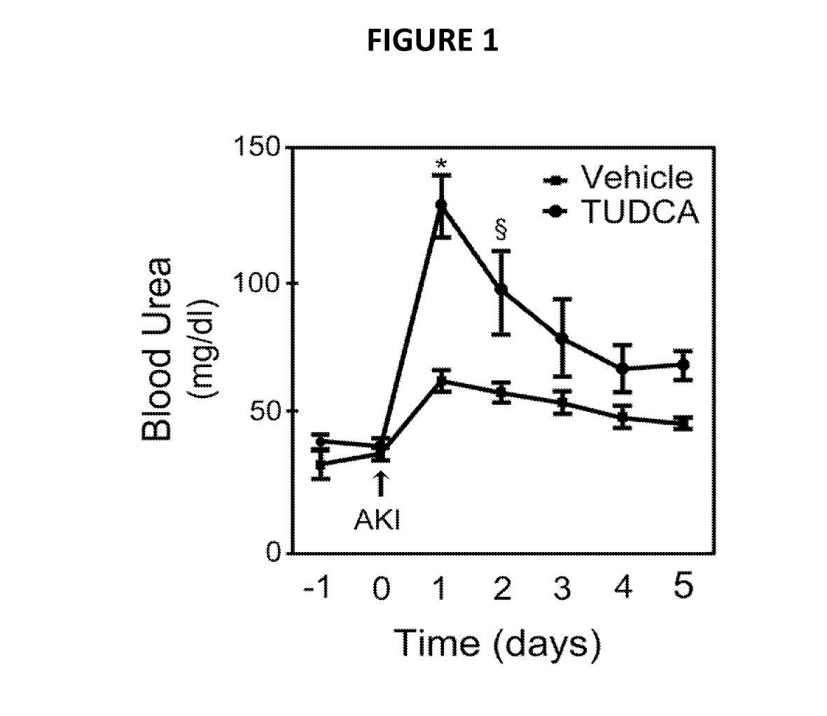

Functional protection: Rats were given 400 mg/kg/day of TUDCA or equal volume of vehicle from three days before until five days following the induction of AKI. Renal function was determined by daily measurements of blood urea levels. Rats in the TUDCA group had significantly less elevation in blood urea levels on days 1 (p<0.001) and 2 (p<0.01) following AKI as compared to those in the vehicle group (FIG. 1). Although on days 3-5 blood urea was lower in the TUDCA-treated rats, the difference was not statistically significant. Interestingly, the blood urea continued to decline in the TUDCA-treated group until the day of euthanasia (day 5), while it stabilized above baseline in the vehicle-treated group.

The results demonstrated that daily administration of 400 mg/kg of TUDCA protected against ischemic AKI. Rats that received TUDCA had significantly less elevation in blood urea levels on day 1 and day 2 following ischemia-reperfusion injury as compared to rats that received vehicle (p<0.01). Similarly, on days 3-5, blood urea levels continued to remain lower in the TUDCA-treated rats. Results are expressed as mean ± standard deviation of a least 3 different animals in each group (*p<0.001 and §p<0.01 from vehicle-injected controls).

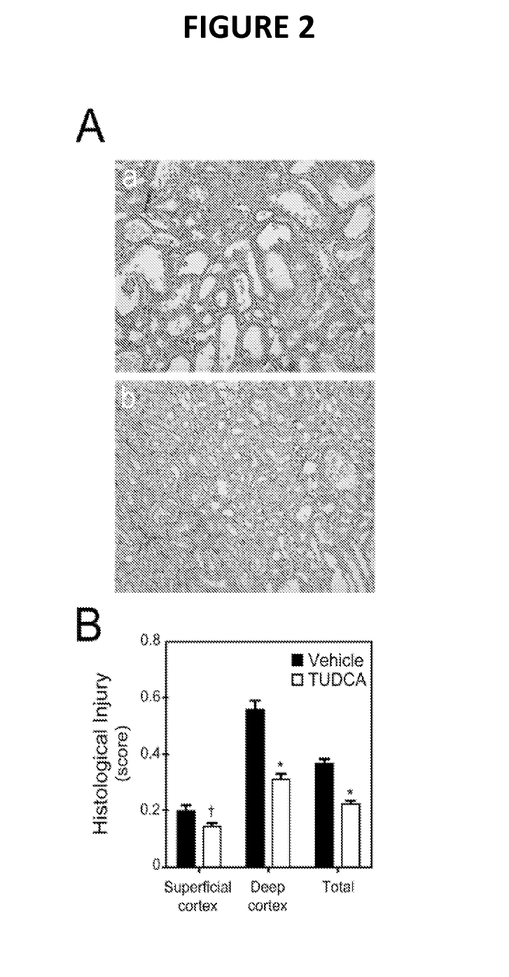

Structural protection: Kidneys were harvested five days following the induction of ischemic AKI. Many proximal tubules from the deep cortex (FIG. 2A, a) had significant injury in vehicle-treated rats; in contrast, TUDCA-treated rats had minimal injury (FIG. 2A, b). There was significantly less injury in the superficial (p<0.05) and deep (p<0.001) cortex in TUDCA-treated rats as compared to the vehicle-treated rats (FIG. 2B). There were no differences in the medulla or papilla (data not shown).

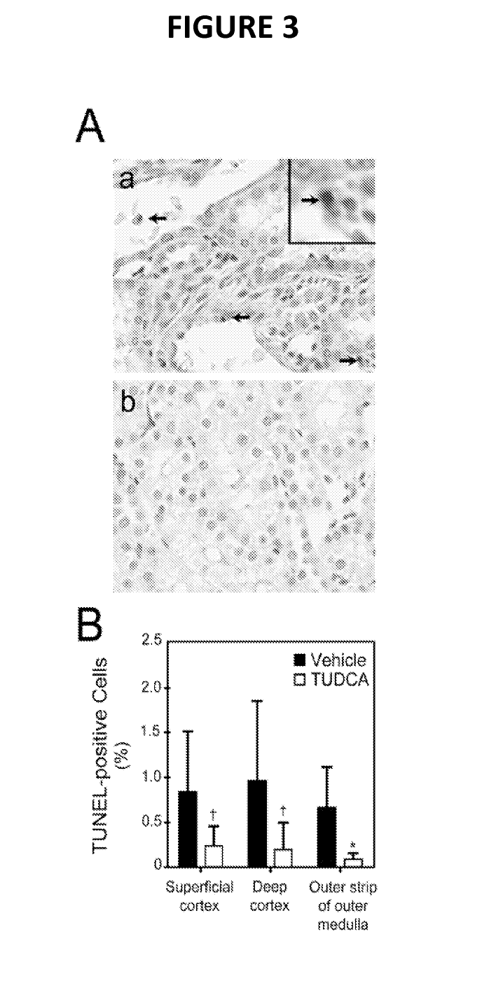

FIG. 2A shows representative PAS stained images from deep cortex from animals that received vehicle control (panel a) or TUDCA (panel b). FIG. 2B is a graph showing that animals that received TUDCA as compared to controls, showed significantly less damage in the deep cortex where the S3 segment is located. Results are expressed as mean ± standard deviation of a least 3 different animals in each group (*p<0.001 and [symbol] p<0.05 from vehicle-injected controls).Protection against apoptosis: As TUDCA is a potent anti-apoptotic molecule, we quantified cells undergoing apoptosis in kidney sections by the transferase mediated deoxyuridine triphosphate (dUTP)-digoxigenin nick-end labeling (TUNEL) assay (FIGS. 3A and B). There were significantly less TUNEL-positive cells in the superficial and deep cortices (p<0.05) and outer strip of the outer medulla (p<0.001) in the TUDCA-treated rats (FIG. 3A, panel a) as compared to the vehicle-treated rats (FIG. 3A, panel b). The apoptotic cells were exclusively limited to the proximal tubules, which were identified by morphology in the periodic acid-Schiff stain (PAS) stained sections.

FIG. 3A shows representative images from cortico-medullary junction from vehicle (panel a; control) and TUDCA-treated (panel b) groups. Brown staining and arrows identify TUNEL-positive cells. FIG. 2B is a graph showing that there were significantly less TUNEL-positive cells in the TUDCA-treated group as compared to the vehicle-treated (control) group in the cortex (p<0.05) and outer strip of the outer medulla (p<0.001). Results are expressed as mean ± standard deviation of a least 3 different animals in each group (*p<0.001 and [symbol] p<0.05 from vehicle-injected controls).

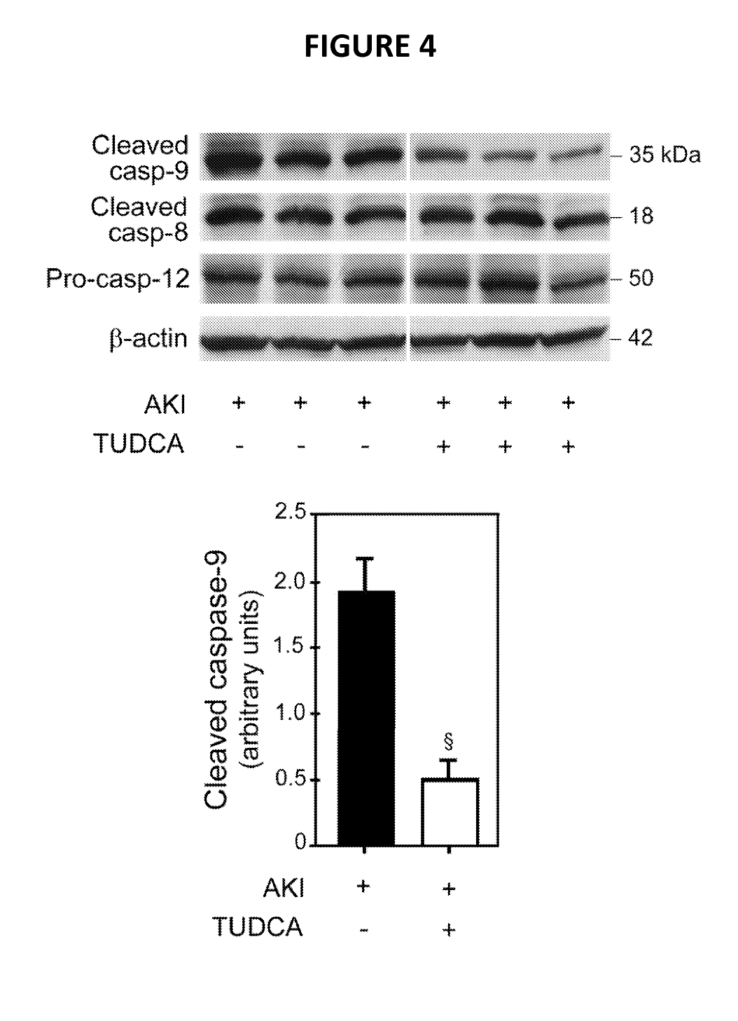

Apoptosis pathway analysis: We determined activation of the mitochondrial, death-receptor, and endoplasmic reticulum (ER)-stress pathway of apoptosis by Western blot analysis for active caspase-9, caspase-8, and caspase-12, respectively. Activation of caspase-9 was significantly inhibited by TUDCA (p<0.01) (FIG. 4); and the results were confirmed by densitometry using β-actin as the loading control. Interestingly, there was no significant difference in the activation of caspase-8 and caspase-12 between the TUDCA- and vehicle-treated rats.

FIG. 4 shows that TUDCA treatment significantly blocked activation of caspase-9 following AKI as compared to vehicle treatment (rats 1, 2, and 3). There was no difference in the activation of caspase-8 and caspase-12 between the TUDCA- and vehicle-treated rats. FIG. 4 also shows a graph illustrating densitometry analysis of cleaved caspase-9 normalized for β-actin. When densitometry results for caspase-9 were compared between the TUDCA- and vehicle-treated groups, there was significantly less (p<0.01) activation of caspase-9 in the TUDCA group. Results are expressed as mean ± standard deviation of a least 3 different animals in each group (§p<0.01 from vehicle-injected controls).

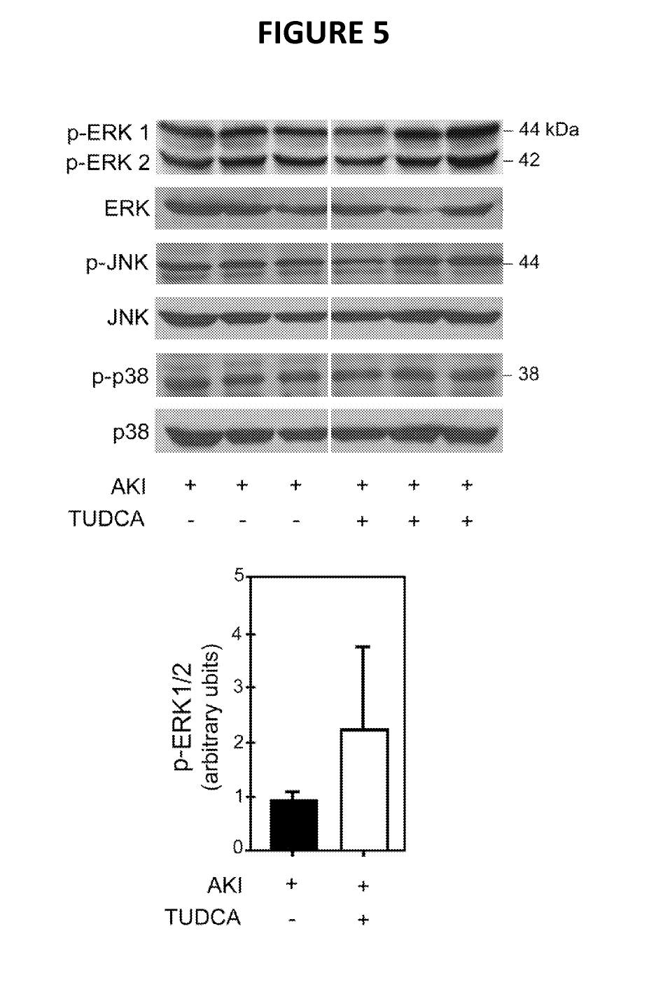

Survival pathway analysis: Extracellular regulated kinase 1/2 (ERK1/2), c-Jun N-terminal kinase (JNK), and p38, which are the key components of mitogen-activated protein kinase (MAPK) survival pathway, were analyzed by Western blot. There was increased activation of ERK1/2 in the TUDCA group, although without statistical significance, when compared with controls (FIG. 5). No difference was detected in the amount of p-JNK and p-p38 between the two groups.

FIG. 5 shows that TUDCA treatment upregulated ERK1/2 following ischemia-reperfusion injury to the kidney in rats. FIG. 5 also shows a graph illustrating densitometry analysis of ERK1/2 in each rat in the TUDCA group were compared with those in the vehicle group; the difference as not significant (p=0.29). Results are expressed as mean ± standard deviation of a least 3 different animals in each group.

### Cell Culture Experiments

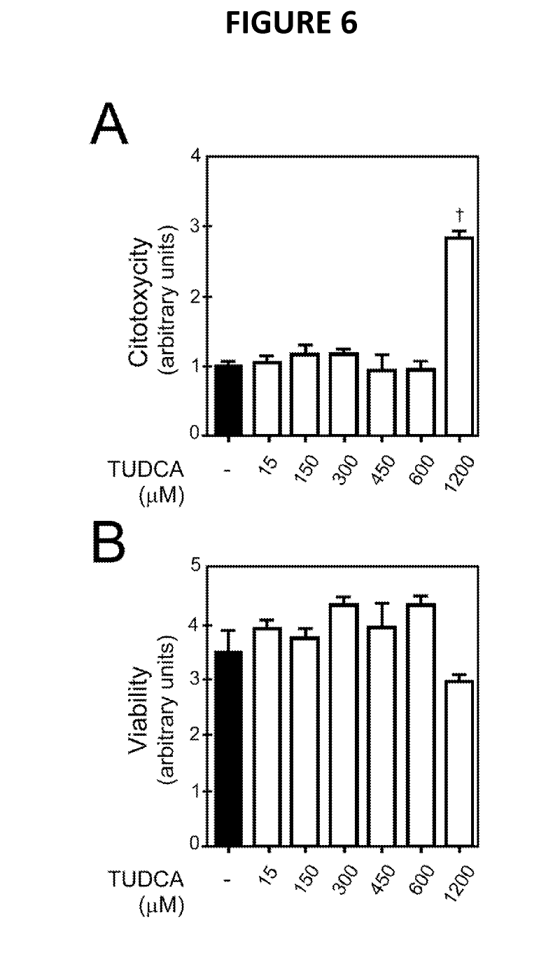

Toxicity studies of TUDCA: We treated human renal proximal tubular epithelial cells (RPTE) cells with different concentrations of TUDCA, and performed cytotoxicity and viability assays. TUDCA in concentrations from 15 to 600 μM did not cause cytotoxicity; only 1200 μM of TUDCA was cytotoxic (p<0.05) (FIG. 6A). None of the tested concentrations of TUDCA decreased cell viability (FIG. 6B). We chose concentrations up to 600 μM of TUDCA for subsequent experiments.

Cells were treated with either vehicle (control) or 15 to 1200 μM of TUDCA for 24 hours. TUDCA was not cytotoxic in concentrations from 15 to 600 μM. Significant cytotoxicity was seen only with 1200 μM of TUDCA, as compared to the vehicle (p<0.05). TUDCA did not significantly decrease cell viability in any of the tested concentrations from 15 μM to 1200 μM. Results are expressed as mean ± standard deviation. All experiments were performed in triplicate ( [symbol] p<0.05 from vehicle-treated to control).

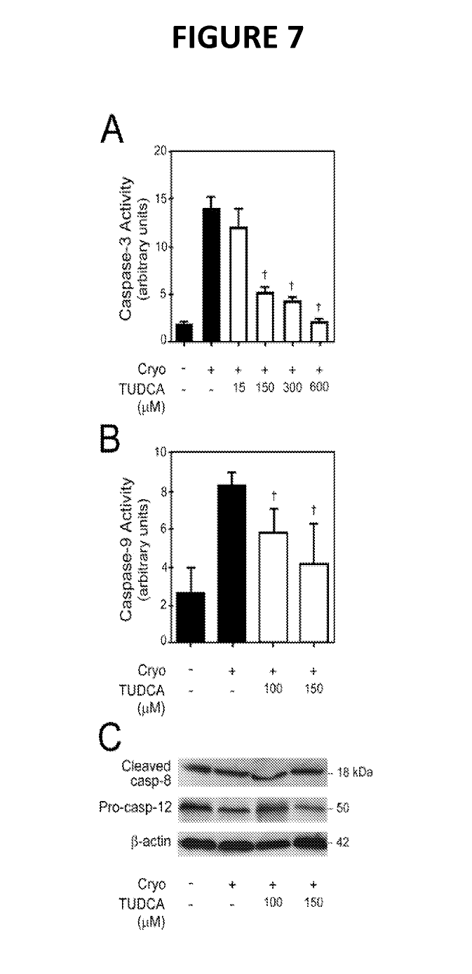

Protection against apoptosis: We determined activation of the final common pathway of apoptosis by caspase-3 activity assay. Cryopreservation injury significantly activated caspase-3 in RPTE cells (p<0.01), which was significantly inhibited by 150-600 μM of TUDCA in a dose-dependent fashion (p<0.05) (FIG. 7A). Activation of the mitochondrial pathway of apoptosis was determined via caspase-9 activity assay (FIG. 7B). Cryopreservation injury consistently activated caspase-9 in RPTE cells (p<0.01), which was significantly inhibited by 100 μM and 150 μM of TUDCA (p<0.05). Next, we determined the activation of death receptor and ER-stress pathways of apoptosis by performing Western blot analysis for caspase-8 and procaspase-12 (FIG. 7C). There was no activation of procaspase-8 and procaspase-12 in our model of cryopreservation injury, and TUDCA had no effect on the activation. Thus, TUDCA protected against cryopreservation injury by inhibiting the mitochondrial pathway of apoptosis.

FIG. 7A shows caspase-3 activity following cryoinjury in RPTE cells treated with either vehicle or different concentrations of TUDCA. Caspase-3 activity in cryoinjured RPTE cells was compared with that in uninjured RPTE cells. FIG. 7B shows caspase-9 activity following cryoinjury in RPTE cells treated with either vehicle or different concentrations of TUDCA. There was statistically significant increased caspase-9 activity in cryoinjured cells as compared to uninjured cells (p<0.05). Both 100 and 150 μM of TUDCA significantly decreased caspase-9 activity. FIG. 7C shows caspase-8 and caspase-12 analysis following cryoinjury in RPTE cells treated with either vehicle or different concentrations of TUDCA. There was no difference in the amount of caspase-8 and procaspase-12 between the uninjured and cryoinjured cells treated with vehicle or TUDCA. Results are expressed as mean ± standard deviation. All experiments were performed in triplicate ( [symbol] p<0.05 from vehicle-treated control).

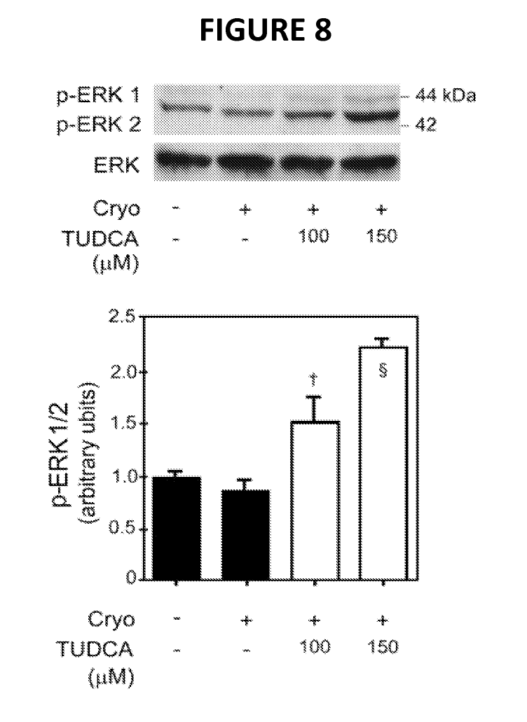

Survival pathway analysis: We performed Western blot analysis for active forms of the MAPKs (ERK, JNK, and p38) to determine activation of survival pathways. Treatment with 100 and 150 μM of TUDCA activated ERK1/2 (FIG. 8; p<0.05 and p<0.01, respectively); however, there was no effect of TUDCA on JNK or p38 (data not shown). Thus, upregulation of ERK1/2 by TUDCA contributed to protection against cryopreservation injury.

FIG. 8 shows that TUDCA treatment upregulated ERK1/2 following cryoinjury to primary human RPTE cells. Phosphorylated ERK1/2 protein in cryoinjured cells that were treated with either vehicle (control) or 100 μM or 150 μM of TUDCA and compared to uninjured cells. There was no difference in the amount of phosphorylated ERK1/2 between uninjured and cryoinjured cells treated with vehicle. FIG. 8 also shows a densitometry analysis of phosphorylated ERK1/2. Results are expressed as mean ± standard deviation. All experiments were performed in triplicate (§p<0.01 and [symbol] p<0.05 from vehicle-treated control).

While the invention has been described with reference to exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiments disclosed, but that the invention will include all embodiments falling within the scope of the appended claims.

1. A method of treating or preventing kidney injury comprising administering to a patient an effective amount of bile acid, a salt thereof, an analog thereof, or a combination thereof.

2. The method of claim 1, wherein the bile acid is tauroursodeoxycholic acid (TUDCA).

3. The method of claim 1, wherein the patient is a human patient.

4. The method of claim 1, wherein the bile acid, a salt thereof, an analog thereof, or a combination thereof is administered in combination with a pharmaceutical-grade carrier.

5. The method of claim 1, wherein the bile acid, a salt thereof, an analog thereof, or a combination thereof is administered intravenously, parenterally, orally or intramuscularly.

6. The method of claim 1, wherein the administration of any therapy would spontaneously or eventually result in the synthesis of TUDCA or any derivative or any analog or any precursor or salt of TUDCA in the patient.

7. The method of claim 1, wherein the administration of any diet would spontaneously or eventually result in the synthesis of TUDCA or any derivative or any analog or any precursor or salt of TUDCA in the patient.

8. The method of claim 1, wherein the administration of any therapy would spontaneously alter the endogenous level of TUDCA or any derivative or any analog or any precursor or salt of TUDCA in the patient.

9. The method of claim 1, wherein the administration of any diet would spontaneously alter the endogenous level of TUDCA or any derivative or any analog or any precursor or salt of TUDCA in the patient.

10. The method of claim 1, wherein the insertion of any gene into the patient would spontaneously alter the levels of TUDCA or any derivative or any analog or any precursor or salt of TUDCA in the patient.

11. The method of claim 1, wherein the administration of foreign DNA into the patient would spontaneously or eventually result in the synthesis of TUDCA or any derivative or any analog or any precursor or salt of TUDCA in the patient.

12. A method for treating a human patient with acute kidney injury of any form, the method comprising administering to a patient an effective amount of a compound selected from the group consisting of TUDCA, a salt thereof, an analog thereof, and a combination thereof;

13. The method of claim 12, wherein the compound is an analog of TUDCA and is unconjugated ursodeoxycholic acid (UDCA);

14. The method of claim 12, wherein the compound comprises of any analog or precursor or derivative or salt of TUDCA, including UDCA.

15. Use of tauroursodeoxycholic acid (TUDCA), a salt thereof or an analog thereof, for treatment of kidney injury.

16. Use of tauroursodeoxycholic acid (TUDCA), a salt thereof or an analog thereof, in the manufacture of a medicament for treatment of kidney injury.

17. Tauroursodeoxycholic acid (TUDCA) for use in the treatment of kidney injury or to kidney disease.
